(19) United States
(12) Patent Application Publication
(10) Pub. No.: US 2017/0056257 A1
(43) Pub. Date: Mar. 2, 2017

(54) BELTED STRUCTURE

(71) Applicant: The Procter & Gamble Company, Cincinnati, OH (US)

(72) Inventors: Masaharu NISHIKAWA, Cincinnati, OH (US); Urmish Popatlal Dalal, Milford, OH (US)

(21) Appl. No.: 15/236,522

(22) Filed: Aug. 15, 2016

Related U.S. Application Data
(60) Provisional application No. 62/210,635, filed on Aug. 27, 2015.
(60) Provisional application No. 62/336,777, filed on May 16, 2016.

(51) Int. Cl. A61F 13/49

(52) U.S. Cl. CPC A61F 13/49011; A61F 13/49014

(57) ABSTRACT

Pull-on disposable absorbent articles comprising an elastomeric belt comprising bicomponent nonwovens.

### FIELD

The present disclosure generally relates to pull-on disposable absorbent articles comprising an elastomeric belt and central chassis.

### BACKGROUND

A particular type of absorbent article pant design currently marketed is sometimes called the “balloon” pant. The balloon pant design usually includes a central absorbent chassis and an elastic belt. The elastic belt is usually relatively wide (in the longitudinal direction) and elastically stretchable in the lateral direction. It entirely encircles the wearer's waist, and thereby covers a relatively large amount of the wearer's skin, and also makes up a relatively large portion of the visible outside surfaces of the pant. The central chassis portion is typically joined to the inside of the belt in the front, wraps under the wearer's lower torso between the legs, and is joined to the inside of the belt in the rear. As such, balloon pants are a compilation of separate article components. Because of the manner in which absorbent article components are incorporated, even the viewable surfaces of the article may have seams or areas of overlap or connection. Thus, it can be challenging to create an absorbent article wherein the materials that are bonded together form strong and unobtrusive bonds. Therefore, any improvement in the materials of the article components can help form strong bonds and enhance the article's appearance.

### BRIEF DESCRIPTION OF THE DRAWINGS

FIG. 1 is a perspective view of one example of a belted article.

FIG. 2 is a schematic plan view of a belted article precursor structure, prior to joining of the front and rear sections of the belt.

FIGS. 3A-3C are varying longitudinal cross-section views taken at line 3-3 of FIG. 2.

FIG. 4 is a longitudinal cross-section view taken at line 4-4 of FIG. 2.

FIG. 5 is a schematic top view of a belted article with flange side seams.

FIG. 6 is a schematic top view of a belted article with overlapping side seams.

FIGS. 7A-7D are cross-section views of various waist edge configurations of belted articles.

### DETAILED DESCRIPTION

Various non-limiting embodiments of the present disclosure will now be described to provide an overall understanding of the principles of the structure, function, manufacture, and use of the absorbent articles disclosed herein. One or more examples of these non-limiting embodiments are illustrated in the accompanying drawings. Those of ordinary skill in the art will understand that the absorbent articles described herein and illustrated in the accompanying drawings are non-limiting example embodiments and that the scope of the various non-limiting embodiments of the present disclosure are defined solely by the claims. The features illustrated or described in connection with one non-limiting embodiment may be combined with the features of other non-limiting embodiments. Such modifications and variations are intended to be included within the scope of the present disclosure.

The following term explanations may be useful in understanding the present disclosure:

“Absorbent article” refers to pull-on garments generally worn by infants and other incontinent individuals to absorb and contain urine, feces and/or menses. It should be understood, however, that the term absorbent article is also applicable to other garments such as training pants, incontinent briefs, feminine hygiene garments or panties, and the like. In some embodiments, “absorbent article” may refer to a taped diaper.

The terms “elastic,” “elastomer,” and “elastomeric” refer to a material which generally is able to extend to a strain of at least 50% without breaking or rupturing, and is able to recover substantially to its original dimensions after the deforming force has been removed.

As used herein, “graphic” refers to formation of an object, which may or may not be colored. A graphic, however, does not include a field of color alone, wherein no formation of an object exists.

“Lateral”, with respect to a pant and its wearer, refers to the direction generally perpendicular with the wearer's standing height, or the horizontal direction when the wearer is standing. “Lateral” is also the direction generally perpendicular to a line extending from the midpoint of the front waist edge to the midpoint of the rear waist edge.

“Longitudinal”, with respect to a pant and its wearer, refers to the direction generally parallel with the wearer's standing height, or the vertical direction when the wearer is standing. “Longitudinal” is also the direction generally parallel to a line extending from the midpoint of the front waist edge to the midpoint of the rear waist edge.

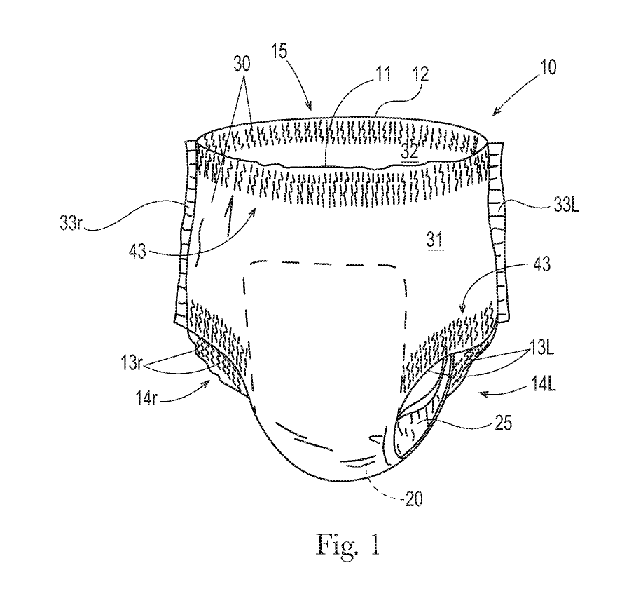

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and, preferably, to be recycled, composted or otherwise disposed of in an environmentally compatible manner). The pull-on garment is also preferably “absorbent” to absorb and contain the various exudates discharged from the body. A preferred embodiment of the absorbent article is the disposable absorbent pull-on garment, shown in FIG. 1.

The term “substrate” is used herein to describe a material that is primarily two-dimensional (i.e., in an XY plane) and whose thickness (in a Z direction) is relatively small (i.e. 1/10 or less) in comparison to its length (in an X direction) and width (in a Y direction). Non-limiting examples of substrates include a web, layer or layers of fibrous materials, nonwovens, and films and foils, such as polymeric films or metallic foils, for example. These materials may be used alone or may comprise two or more layers laminated together. As such, a web may be a substrate or may be a laminate of two or more substrates.

### Article

Many existing absorbent pants are structured such that a backsheet and topsheet of a central chassis structure extend to, and from, the front and rear waist edges of the pant in the regions near the wearer's navel in the front, and small of the back in the rear. Separate and discrete side/hip panels are joined to longitudinal (side) edges of the central chassis structure in its front and rear regions, joining them to form the pant structure.

An alternate configuration for absorbent pants is one in which the central chassis structure does not extend to, or form, the front and rear waist edges of the pant. Rather, an elasticized belt structure entirely encircles the wearer's waist and forms the waist edge about the entire pant, and the side/hip panels. The central chassis is joined to the belt structure, usually on the inside thereof, with its ends disposed at locations in the front and rear waist regions somewhat below the waist edges of the belt structure. The elastic belt is usually relatively wide (in the longitudinal direction) and elastically stretchable in the lateral direction. It entirely encircles the wearer's waist, and thereby covers a relatively large amount of the wearer's skin. This configuration is sometimes known as a “belt” or “balloon” configuration (hereinafter, “belt” configuration).

FIG. 1 is a general simplified perspective depiction of a disposable absorbent pant 10 having a belt configuration. Pant 10 may include a central chassis 20 and a belt structure 30. Belt structure 30 may be elastically extensible in the lateral direction, providing elastic stretchability for ease of donning, and a snug and comfortable fit following donning. Central chassis 20 may include a wearer-facing, liquid permeable topsheet (not specifically shown in FIG. 1), an outer- or garment-facing backsheet (not specifically shown in FIG. 1) and an absorbent core (not specifically shown in FIG. 1) sandwiched or enveloped between the topsheet and backsheet. A pair of laterally opposing, longitudinally extending barrier cuffs 25 also may be included with the central chassis in a crotch region thereof, disposed adjacent to the topsheet. Generally the central chassis and barrier cuffs may have any construction and components, including leg cuff structures, suitable for disposable diapers, training pants, and adult incontinence pants, such as, but not limited to, those described in U.S. Pat. No. 8,939,957 and application(s) claiming priority thereto. Belt structure 30 may have a front portion 31 and a rear portion 32. Front and rear portions 31, 32 may be joined together at respective left and right side seams 33l, 33r. Belt structure 30 may form front and rear waist edges 11, 12 defining waist opening 15, and at least portions of left and right leg opening edges 13l, 13r of the pant 10. As shown in the FIG. 1, the portions of left and right leg opening edges 13l, 13r that the belt structure 30 forms may follow curvilinear paths. Alternatively, the portions of left and right leg opening edges 13l, 13r that the belt structure 30 forms may be substantially straight.

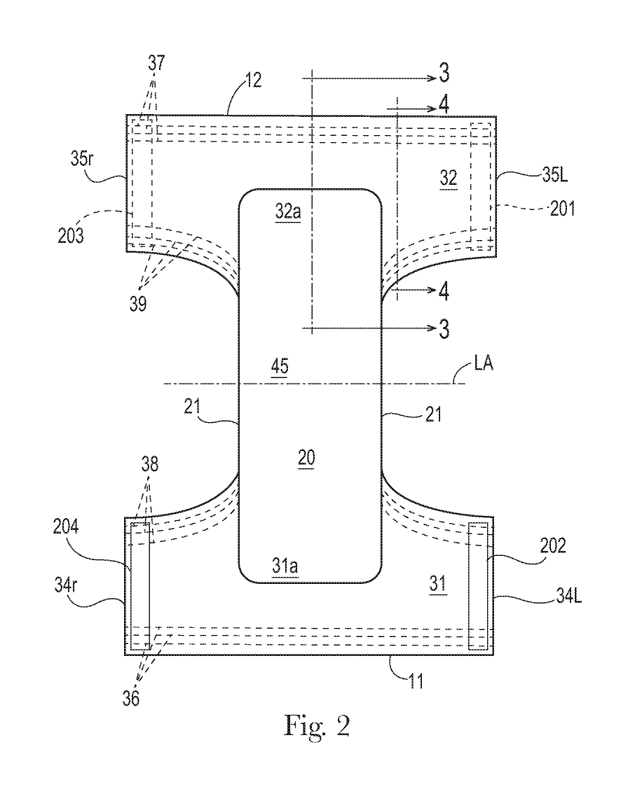

FIG. 2 is a simplified plan view of the precursor structure of the pant 10 shown in FIG. 1, shown prior to joining of front and rear portions 31, 32 along their respective side edges 34l, 35l and 34r, 35r. Front region 31a, including front portion 31, and rear region 32a, including rear portion 32, may each include anywhere from 25 percent to 40 percent of the overall longitudinal length of the precursor structure; correspondingly, a crotch region 45 may include anywhere from 20 percent to 50 percent of the overall longitudinal length of the precursor structure, with at least a portion thereof lying at lateral axis LA. The length of the side edges 34l and 34r may be about equal to the length of the side edges 35l and 35r respectively. The length of the side edges 34l and 34r may be substantially shorter than the length of the side edges 35l and 35r respectively. To form pant 10, the precursor structure may be folded along lateral axis LA to bring front and rear regions 31a, 32a, and front and rear portions 31, 32 together such that their side edges 34l, 35l and 34r, 35r, respectively, may be joined at seams 33l, 33r (as shown in FIG. 1). Seams 33l, 33R may be formed by adhesive, thermal, pressure, or ultrasonic bonding, and combinations thereof. The length of the seams 33l, 33R may be about equal to the length of the side edges 34l, 35l, 34r and 35r. The length of the seams 33l, 33R may be substantially shorter than the length of the side edges 34l, 34r, 35l or 35r. In an alternative example, the seams may be formed by mechanical fasteners such as cooperating pairs of hook-and-loop fastening components disposed along side edges 34r, 35r and 34l, 35l. Fasteners may also include tape tabs, interlocking fasteners such as tabs & slots, buckles, buttons, snaps, and/or hermaphroditic fastening components. Exemplary surface fastening systems are disclosed in U.S. Pat. Nos. 3,848,594; 4,662,875; 4,846,815; 4,894,060; 4,946,527; 5,151,092; and 5,221,274, while an exemplary interlocking fastening system is disclosed in U.S. Pat. No. 6,432,098. The fastening system may also include primary and secondary fastening systems, as disclosed in U.S. Pat. No. 4,699,622. Additionally exemplary fasteners and fastener arrangements, the fastening components forming these fasteners, and the materials that are suitable for forming fasteners are described in U.S. Published Application Nos. 2003/0060794 and 2005/0222546 and U.S. Pat. No. 6,428,526.

Still referring to FIG. 2, one or both of front and rear portions 31, 32 may include at least a first elastic member 36, 37 disposed nearer the waist edges 11, 12 and at least a second elastic member 38, 39, disposed nearer the leg opening edges 13l, 13r. As suggested in FIG. 2, one or a plurality of waist elastic members 36, 37 may be disposed in a substantially straight lateral orientation, and one or a plurality of leg elastic members 38, 39 may be disposed along curvilinear paths to provide hoopwise elastic stretch about the leg openings 13l, 13r (as shown in FIG. 1). Alternatively, leg elastic members 38, 39 may be disposed in a substantially straight lateral orientation near the leg openings 13l, 13r. For purposes of manufacturing a pant having a neat appearance as will be described below, it may be desired that leg elastic members 38, 39 terminate proximate the respective longitudinal edges 21 of chassis 20. For purposes herein, where used to describe a positional relationship between two features, “proximate” is intended to mean within 2.0 cm, more preferably within 1.0 cm, of the identified features.

Elastic members 36, 37, 38 and 39 may be in the form of film or sections or strips thereof, strips, ribbons (flat strands), bands or strands of circular or any other cross-section, formed in any configuration of any elastomeric material such as described in, for example, co-pending U.S. applications Ser. Nos. 11/478,386 and 13/331,695, and U.S. Pat. No. 6,626,879. A suitable example is LYCRA HYFIT strands, a product of Invista, Wichita, Kans. The elastic strands can have a cross section perpendicular to the strand longitudinal axis that is substantially non-circular. Substantially non-circular means that the ratio of the longest axis of the cross section to the shortest axis of the cross section is at least about 1.1. The ratio of the longest axis of the cross section to the shortest axis of the cross section can be about 1.1, about 3.0, about 5.0, about 10.0, or about 50.0. In some embodiments, this ratio can be at least about 1.1, or at least about 3.0. The shape of the cross section perpendicular to the strand longitudinal axis of the substantially non-circular strands can be rectangular (e.g., with rounded corners) which are also referred to as “flat” strands, trilobal, or oblong (e.g., oval) in the cross section. These substantially non-circular strands can provide more surface area to bond with nonwoven fabrics than the strands that are circular in cross section. Such an increase in surface area can increase the bond strength between the elastomeric strands and nonwoven. Flat elastic strands can be made of Spandex, rubber, elastic polyolefins, styrenic block copolymers, thermoplastic polyurethane, thermoplastic polyester, polyether block amide or any combination of them. Thermoplastic polyurethane tapes available from Fulflex such as Clear-Fit™ can be used as flat elastic strands.

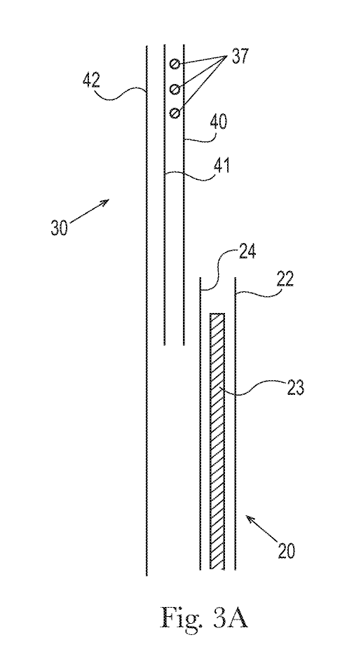

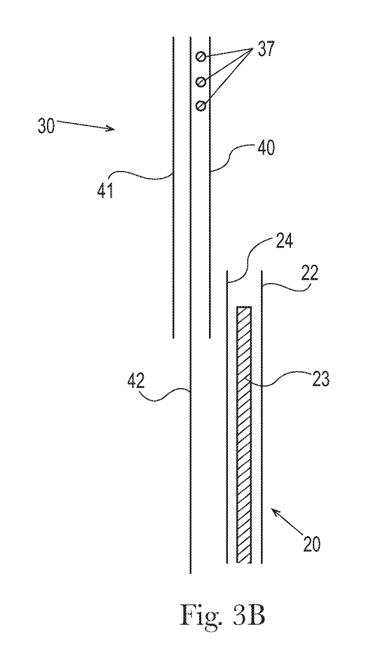

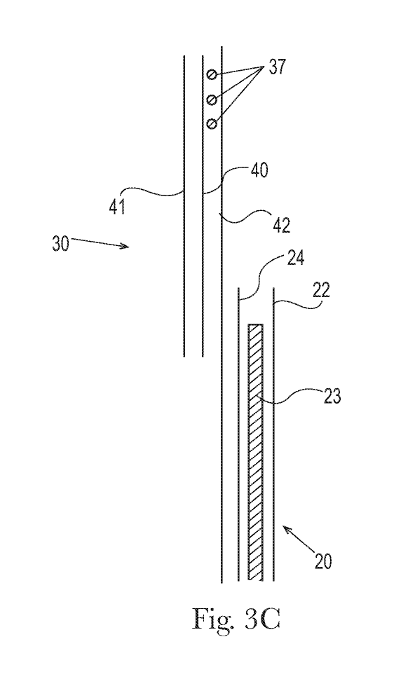

FIGS. 3A-3C are examples of potential longitudinal cross-sections taken at line 3-3 through the rear portion 32 of the belt structure and rear region of the pant as shown in FIG. 2, depicting features in three possible configurations. It can be appreciated that in each of these particular examples, the cross-section may substantially mirror a cross-section taken through the front portion 31 of the belt structure and the front region of the pant.

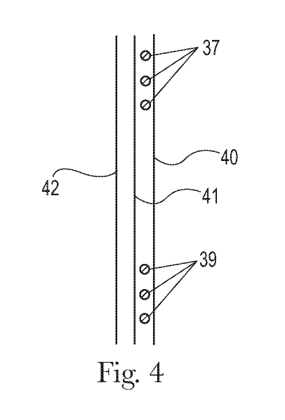

FIG. 4 is an example of a potential longitudinal cross-section taken at line 4-4 through the rear portion 32 of the belt structure and rear region of the pant as shown in FIG. 2, depicting features in one configuration. It can be appreciated that this cross-section may also be a substantial mirror image of a cross-section taken through the front portion 31 of the belt structure and the front region of the pant. Belt structure 30 where shown in FIG. 4 has the same layers and components as those depicted in FIG. 3A, but with the addition of leg elastic members 39 and without the chassis components, as a result of the location of the cross-section. As suggested in FIG. 2, leg elastic members 39 may terminate proximate the longitudinal edges 21 of central chassis 20; thus, they do not appear in FIGS. 3A and 3B. Additional elastics (not shown) may be disposed longitudinally between the waist elastics and the leg elastics.

Referring to FIGS. 3A-3C, chassis 20 may have liquid permeable topsheet 22 forming at least a portion of its inner, wearer-facing surface. Topsheet 22 may be formed of a nonwoven web material which is preferably soft and compatible with sensitive skin, and may be formed of and have any of the features of topsheets used in disposable diapers, training pants and inserts including those described in, for example, U.S. application Ser. No. 12/841,553. Chassis 20 may also have an outward-facing backsheet 24, which may be liquid impermeable. Backsheet 24 may be formed of and have any of the features of backsheets used in disposable diapers and training pants including those described in, for example, the U.S. patent application referenced immediately above. Chassis 20 may also have an absorbent core 23 disposed between topsheet 22 and backsheet 24. Absorbent core 23 may include one or more absorbent acquisition, distribution and storage material layers and/or components; it may be formed of and have any of the features of absorbent cores used in disposable diapers and training pants including those described in, for example, the U.S. patent application referenced immediately above.

As suggested in FIGS. 3A-3C, chassis 20 may be affixed to a belt structure 30, to the inner, wearer-facing side thereof, or alternatively, to the outer, garment-facing surface thereof. Chassis 20 may be bonded to the belt structure 30 by adhesive, by thermal bonds/welds, mechanical fasteners or a combination thereof.

Referring to FIGS. 3A and 4, belt structure 30 may have a first belt layer 40 (or inner belt) which may be formed of a suitable nonwoven web material. Since the first belt layer may come into direct contact with the wearer's skin, it may be deemed preferable to select a nonwoven web material for the layer that is soft, comfortable and relatively breathable/vapor permeable. One or more waist elastic members 37 may be disposed between first belt layer 40 and a second belt layer 41 (also called the outer belt). Second belt layer 41 may be formed of the same, similar or differing nonwoven web material as first belt layer 40. First belt layer 40 and second belt layer 41 may be bonded together by adhesive, a pattern of thermal bonds or a combination thereof, such that first belt layer 40 and second belt layer 41 form a laminate, with the one or more waist elastic members 37 sandwiched and affixed there between. Similarly, referring to FIG. 4, the one or more leg elastic members 39 may be affixed and sandwiched between first belt layer 40 and second belt layer 41.

Also as shown in FIGS. 3A-3C and 4, the belt structure may include a longitudinally extending wrapping layer 42. Wrapping layer 42 may form a layer that wraps not only about the front and rear portions of the belt structure, but also extends from the front portion, around and beneath the chassis 20 through the crotch region, and into the rear portion. The wrapping layer 42 may be formed of a single material web disposed as a layer of the rear portion 32 of the belt structure 30, the central chassis 20, and the front portion 31 of the belt structure 30. Wrapping layer 42 may be disposed so as to form an outer layer or outer cover of the belt structure 30 in the front and rear portions as suggested in FIGS. 3A and 4, an intermediate layer in the front and rear portions as suggested in FIG. 3B, or an inner layer as suggested in FIG. 3C. Wrapping layer 42 may be formed of any suitable nonwoven web material having desired properties of softness and mechanical strength. Wrapping layer 42 is optional. That is, there are embodiments in which there is no wrapping layer and the rear portion 32 of the belt and the front portion 31 of the belt, when the article is laid out flat, may be discrete and without a common layer. The only part connecting the two belts in such embodiments would be the center chassis.

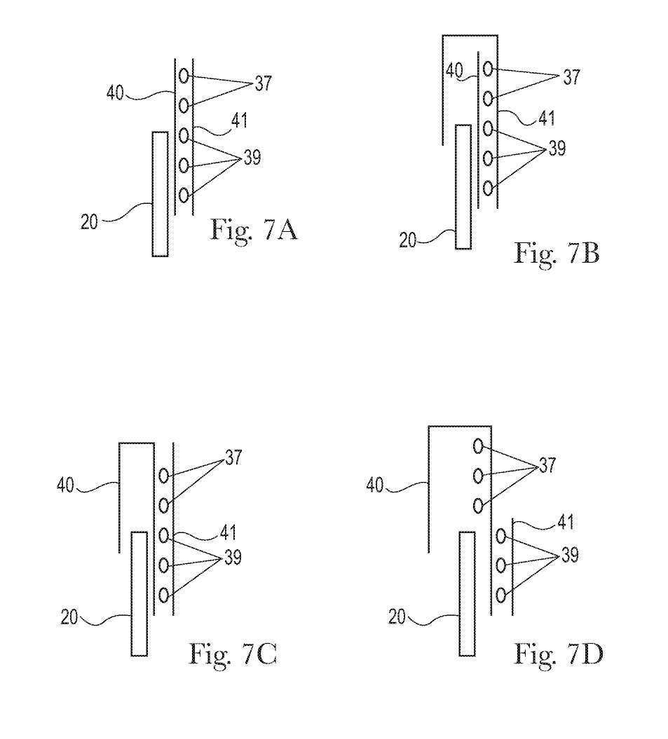

In some embodiments, the inner belt nonwoven 40 and the outer belt nonwoven 41 end at waist edge, such as is depicted in a cross-section view of an article in FIG. 7A. In other embodiments, such as FIG. 7B, the outer belt nonwoven 41 is extended up and folded over the inside of the central chassis 20. In this embodiment, the edge of the inner belt nonwoven at the waist edge may be aligned to or away from the folding that the outer belt nonwoven forms. In some cases, the inner belt nonwoven may be shortened such that it extends towards the waist only as far as the outer belt nonwoven extends away from the waist after being folded over the inside of the central chassis. That is, in FIG. 7B, in some cases, the inner belt nonwoven 40 may be about the same height as the outer belt nonwoven 41 that is on the inside of the central chassis. In some cases, the inner belt nonwoven 40 will extend towards the waist from about 0 mm to about 10 mm further than the folded over end of the outer belt nonwoven 41. In yet another embodiment as shown in FIG. 7C, the outer belt nonwoven may not be folded over at the waist, even though the inner belt nonwoven 40 is folded over the central chassis. In this embodiment, the edge of the outer belt nonwoven at the waist edge may be aligned to the folding that the inner belt nonwoven forms. FIG. 7D shows another embodiment, in which the outer belt nonwoven 41 extends towards the waist only as far as the central chassis. The outer belt nonwoven does not extend as far as where the inner belt nonwoven 40 is folded at the waist. The waist elastic members 37 are between the layers of the folded inner belt nonwoven 40.

In some embodiments, when the belt configuration is such as is shown in FIG. 7B, there may be an additional cuff, a waist cuff. The waist cuff may extend, for example, from the end of the outer belt nonwoven 41 into the inside of the article. The waist cuff can provide additional protection against excrement escaping the center of the article towards the waistband. Such a cuff may be, for example, two nonwovens with elastic strands in between. The additional cuff may be a discrete cuff attached to the belt or it may be an extension of the belt itself. Examples of such a waist cuff can be found in WO 2010/109866 and JP 04354948, particularly FIG. 3 of JP 04354948.

Examples of suitable nonwoven web materials useful for forming any of layers 40, 41 and 42 are described in U.S. application Ser. No. 13/090,761. Some examples described above, as well as other examples not expressly described, may also be advantageous because they may lend themselves to relatively efficient manufacture.

In general terms, the belt structure comprises an inner layer that is in contact with the wearer's skin when the article is worn. This inner layer may be formed of an inner nonwoven web comprising an inner surface. According to different embodiments described above, the inner surface that is in contact with the wearer's skin may be the first belt layer (inner belt) (for example, see FIG. 3A) or may be the wrapping layer (for example, see FIG. 3C). The belt structure also comprises an outer layer formed of an outer nonwoven web comprising an outer surface. This outer surface is the outermost surface of the article. In some embodiments described above, the outer surface may be the wrapping layer (for example, see FIG. 3A) or may be the second belt layer (outer belt) (for example, see FIG. 3C).

In some embodiments, the rear belt portion may be offset from the front belt portion, i.e., the rear belt portion may have a longer longitudinal length than the front belt portion longitudinal length to allow better coverage on the wearer, such as is described in U.S. filing Ser. No. 11/197,203.

The article, in some cases the chassis, may have a liquid permeable topsheet forming at least a portion of its inner, wearer-facing surface. The topsheet may be formed of a nonwoven web material which is preferably soft and compatible with sensitive skin, and may be formed of and have any of the features of topsheets used in disposable diapers, training pants and inserts including those described in, for example, U.S. application Ser. No. 12/841,553. The chassis 20 may also have an outward-facing backsheet, which may be liquid impermeable. The backsheet may be formed of and have any of the features of backsheets used in disposable diapers and training pants including those described in, for example, the U.S. patent application referenced immediately above. Chassis 20 may also have an absorbent core disposed between the topsheet and backsheet. The absorbent core may include one or more absorbent acquisition, distribution and storage material layers and/or components; it may be formed of and have any of the features of absorbent cores used in disposable diapers and training pants including those described in, for example, the U.S. patent application referenced immediately above. The belt structure may be formed of layers of nonwoven web which respectively form inner and outer layers of the belt and the layers of nonwoven web may sandwich one or more elastic members such as a plurality of strands of an elastomeric material. Suitable nonwoven web materials and suitable elastic materials that may be useful in the present invention include those described in U.S. Ser. No. 14/726,812. As suggested in FIG. 2, the chassis 20 may be affixed to a belt structure 30, to the inner, wearer-facing side thereof. Chassis 20 may be bonded to the belt structure 30 by adhesive, by thermal bonds/welds, mechanical fasteners or a combination thereof. The belt structure may be referred to as flaps. Some of these article components are discussed in more detail below.

### Belt Nonwovens

The fibrous structures of the present invention may be made by any suitable process known in the art.

Nonwoven webs can be formed by direct extrusion processes during which the fibers and webs are formed at about the same point in time, or by preformed fibers which can be laid into webs at a distinctly subsequent point in time. Example direct extrusion processes include but are not limited to: spunbonding, spunlaid, meltblowing, solvent spinning, electrospinning, carded, film fibrillated, melt-film fibrillated, air-laid, dry-laid, wet-laid staple fibers, and combinations thereof typically forming layers.

As used herein, the term “spunbonded fibers” refers to small diameter fibers, which are formed by extruding molten thermoplastic material as filaments from a plurality of fine, usually circular capillaries of a spinneret. Spunbond fibers are quenched and generally not tacky when they are deposited onto a collecting surface. Spunbond fibers are generally continuous.

As used herein, the term “meltblown fibers” means fibers formed by extruding a molten thermoplastic material through a plurality of fine, usually circular, die capillaries as molten threads or filaments into converging high velocity gas (e.g. air) streams, which attenuate the filaments of molten thermoplastic material to reduce their diameter. Thereafter, the meltblown fibers are carried by the high velocity gas stream and are deposited on a collecting surface to form a web of randomly disbursed meltblown fibers.

Example “laying” processes include wetlaying and drylaying. Example drylaying processes include but are not limited to airlaying, carding, and combinations thereof typically forming layers. Combinations of the above processes yield nonwovens commonly called hybrids or composites. Example combinations include but are not limited to spunbond-meltblown-spunbond (SMS), spunbond-carded (SC), spunbond-airlaid (SA), meltblown-airlaid (MA), and combinations thereof, typically in layers. Combinations which include direct extrusion can be combined at about the same point in time as the direct extrusion process (e.g., spinform and coform for SA and MA), or at a subsequent point in time. In the above examples, one or more individual layers can be created by each process. For instance, SMS can mean a three layer, ‘sms’ web, a five layer ‘ssmms’ web, or any reasonable variation thereof wherein the lower case letters designate individual layers and the upper case letters designate the compilation of similar, adjacent layers. The fibers in a nonwoven web are typically joined to one or more adjacent fibers at some of the overlapping junctions. This includes joining fibers within each layer and joining fibers between layers when there is more than one layer. Fibers can be joined by mechanical entanglement, by chemical bond or by combinations thereof.

In some embodiments, nonwoven fabric can be unbonded nonwoven webs, electrospun nonwoven webs, flashspun nonwoven webs (e.g., TYVEK™ by DuPont), or combinations thereof. These fabrics can comprise fibers of polyolefins such as polypropylene or polyethylene, polyesters, polyamides, polyurethanes, elastomers, rayon, cellulose, copolymers thereof, or blends thereof or mixtures thereof. The nonwoven fabrics can also comprise fibers that are homogenous structures or comprise bicomponent structures such as sheath/core, side-by-side, islands-in-the-sea, and other bicomponent configurations. For a detailed description of some nonwovens, see “Nonwoven Fabric Primer and Reference Sampler” by E. A. Vaughn, Association of the Nonwoven Fabrics Indus-3d Edition (1992).

In some examples, suitable non-woven fiber materials may include, but are not limited to polymeric materials such as polyolefins, polyesters, polyamide, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392 to Land, the disclosure of which is incorporated by reference herein. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. No. 6,645,569 to Cramer et al.; U.S. Pat. No. 6,863,933 to Cramer et al.; and U.S. Pat. No. 7,112,621 to Rohrbaugh et al.; and in co-pending U.S patent application Ser. Nos. 10/338,603 and 10/338,610 by Cramer et al.; and Ser. No. 13/005,237 by Lu et al., the disclosures of which are incorporated by reference herein. The nonwoven fabrics can include fibers or can be made from fibers that have a cross section perpendicular to the fiber longitudinal axis that is substantially non-circular. Substantially non-circular means that the ratio of the longest axis of the cross section to the shortest axis of the cross section is at least about 1.1. The ratio of the longest axis of the cross section to the shortest axis of the cross section can be about 1.1, about 1.2, about 1.5, about 2.0, about 3.0, about 6.0, about 10.0, or about 15.0. In some embodiments, this ratio can be at least about 1.2, at least about 1.5, or at least about 2.0. These ratios can be, for example, no more than about 3.0, no more than about 6.0, no more than about 10.0, or no more than about 15.0. The shape of the cross section perpendicular to the fiber longitudinal axis of the substantially non-circular fibers can be rectangular (e.g., with rounded corners) which are also referred to as “flat” fibers, trilobal, or oblong (e.g., oval) in the cross section. These substantially non-circular fibers can provide more surface area to bond to the elastomeric fiber than nonwoven fabrics with fibers that are circular in cross section. Such an increase in surface area can increase the bond strength between the elastomeric film and fibers.

### Bicomponent Materials

An approach to improving consumer perceptions of component materials involves forming a nonwoven web of “bicomponent” polymer fibers, by spinning such fibers, laying them to form a batt and then consolidating them by calender-bonding with a pattern, selected to provide visual effects. Such bicomponent polymer fibers may be formed by spinnerets that have two adjacent sections, that express a first polymer from one and a second polymer from the other, to form a fiber having a cross section of the first polymer in one portion and the second polymer in the other (hence the term “bicomponent”). The respective polymers may be selected so as to have differing melting temperatures and/or expansion-contraction rates. These differing attributes of the two polymers, when combined in a side by side or asymmetric sheath-core geometry, cause the bicomponent fiber products to curl in the spinning process, as they are cooled and drawn from the spinnerets. The resulting curled fibers then may be laid down in a batt and calender-bonded in a pattern. It is thought that the curl in the fibers adds loft and fluff to the web, enhancing visual and tactile softness signals.

Nonwoven webs can be made of bicomponent or multi-component fibers. One of the components of the fibers, preferably the outer component, may be a soft polymer, such as polyethylene or elastic polyolefin, elastic polyurethane. For example, in a sheath/core bi-component fiber, the sheath can be made of polyethylene while core can be made of polypropylene. Often, the individual components comprise polyolefins such as polypropylene or polyethylene, or their copolymers, polyesters, thermoplastic polysaccharides or other biopolymers. In some embodiments, a nonwoven may be a PE/PET (polyethylene/polyethylene terephthalate) core/sheath bicomponent material, wherein the core is the PET and the outer sheath is PE.

In articles that have permanent side seams, the bicomponent material for the belt outer nonwoven can lead to a higher quality and softer seam. For example, the polyethylene of the outer sheath has a lower melting point than polypropylene of the core (or of nonwovens made completely from polypropylene). When creating the permanent side seams, other than by adhesive, but either through thermal, pressure, or ultrasonic bonding, or combinations thereof, only enough heat or pressure is required to soften or melt the polyethylene. The polyethylene of the front belt outer nonwoven, for example, can then bond with the polyethylene from the corresponding rear belt outer nonwoven. Thus, a belt made with bicomponent outer nonwoven material can require a lower bonding force to make, yet still require a high bond force to break.

The bicomponent materials also may have less adhesive bleed-through. Adhesive bleed-through is often a problem associated with the bonding of nonwovens, so materials that minimize bleed-through are advantageous, and also may allow lower basis weight nonwovens to be used, or alternatively or in conjunction, allow an increased basis weight of adhesive to be used.

### Other Nonwovens Materials and Treatments

Various efforts have been made to provide or alter features of nonwoven web materials with the objective of enhancing consumer perceptions of the materials. These efforts have included selection and/or manipulation of fiber chemistry, basis weight, loft, fiber density, configuration and size, tinting and/or opacifying, embossing or bonding in various patterns, etc. For example, one approach has involved simply increasing the basis weight of the web, otherwise manufactured through a spunlaid/spunbond process that includes deg. formation of a batt of loose spun fibers and then consolidating by calender-bonding in a pattern. All other variables remaining constant, increasing the basis weight of such a web will have the effect of increasing the number of fibers per unit surface area, and correspondingly, increasing apparent thickness, fiber density and/or loft.

One approach to improving consumer perception of softness of a nonwoven material is described in U.S. Pat. Nos. 5,296,289, 5,626,571, and WO9937839. It is an object of these patents to provide a nonwoven web which has been stretched to provide greater coverage with minimal sacrifices in strength as a result of stretching in the machine direction or the cross direction.

Another approach has involved subjecting the web to a hydroenhancing or hydroengorgement process following by optional calender-bonding, to fluff the fibers and increase caliper and loft. Web can be made of one layer of fiber or multi-layer of fibers. Each layer can be made of same material or different. It is believed that the hydroenhancing/hydroengorgement process increases loft and caliper in a manner that enhances visual and tactile softness signals.

Still another approach involves changing nonwoven bond pattern to improve loft. Calendar bonding the nonwoven fibers with certain bond shape (Patent #US2014088535A1) loft of the nonwoven can be improved. Nonwoven fibers can be mono-component or bi-component.

Sleek or silky feel is often preferred over rough texture. Nonwoven silkiness is often measured using dynamic Coefficient of Friction (CoF). Silky nonowovens exhibit CoF dimensionless number between 0.2-0.5. CoF number reduces as silkiness of the material increases. Various approaches can be used to deliver silky feel. Combining loft with silky feel can improve consumer perception of nonwoven softness.

In another approach, nonwoven web can be made of mono-component fiber. However, the fiber is made of lower modulus polyolefin such as polyethylene, or polymer blend to impart silky soft feel. For example, polypropylene nonwoven can be coarse. However, when blended with elastomeric polypropylene (Vistamaxx™ from Exxon), it can help improve the feel of the fiber.

In another approach, nonwoven web can be made of elastomeric polymer. For example, elastomeric polyolefins are used in fibers spinning and to make nonwoven web. Such webs have a very sleek feel, and elastic properties, that is often desired for consumer products.

In another approach, additives can be added to polymer before spinning fiber. During fiber spinning and subsequent process steps to make the nonwoven web, the additives migrate to the fiber surface to provide a silky feel. Amine and amide based additives are commonly used up to 5%.

In another approach, a sleek chemical finish can be coated on the fibers or nonwoven webs. Chemical finishes based on oil, silicone, esters, fatty acids, surfactant etc. can be employed. Softeners such as anionic, cationic or noionic can also be used to improve drape, and touch. Various coating techniques, like roll coating, screen coating, gravure coating, slot coating, spray coating, can be used to apply finish.

In another approach, nonwoven fiber diameter can be reduced to produce fine fibers and to provide silk like feel. Meltblown fiber is one technology to reduce fiber diameter to less than 20 microns. Alternatively, nanofibers, having a diameter of less than 1 micron, made from a melt film fibrillation process with a polymer composition disclosed in U.S. Pat. No. 8,835,709 can be used.

Drape, or the bending or pliability of material without any external force or under its own weight are other parameters that affect consumer perceptions about the material. These can be influenced by variety of factors such as fiber chemistry, thickness, nonwoven bond pattern etc. Pliability or Drape is linked to bending stiffness, which is related to inherent elastic modulus and thickness of material. It has proven to be advantageous for the nonwoven fabric to have a minimum and a maximum bending stiffness, since for instance in the use of the nonwoven fabric in contour matching, as in medical and hygiene articles, too stiff a material would be undesirable. Polyolefin resin with lower elastic modulus and/or lower crystallinity enables lower bending stiffness. One can blend lower elastic modulus materials (elastomer) with traditional fiber making polyolefin resin to make lower modulus fibers. Optimizing bonding can also alter the bending stiffness of the web in the direction desired. Bonds with larger aspect ratio of longitudinal dimension to lateral dimension provides better drape in lateral dimension while providing right rigidity and strength for web handling. Another factor affecting drape is the thickness of the web. The thicker the web is, the lower is the flexibility or pliability. Combining right thickness with fiber chemistry or bond pattern, better drape can be achieved while delivering web performance suitable for processing. The nonwoven fabric with a bending stiffness in MD direction in the range of 1-20 mm and in CD direction in the range of 1-15 mm are desired for belt making.

Nonwoven webs used to make product can often be subjected to “activation” process, either before combining with elastic or after combining. The activation or incremental stretching requires nonwoven webs to have extensibility in addition to softness. Nonwoven webs made with high melt flow rate polymers as disclosed in U.S. Pat. No. 8,926,877 patent or similar extensible nonwovens can be used when activation is preferred.

In some embodiments, the nonwovens may be microtextured or corrugated. Disclosure regarding the method and results of such processes may be found in U.S. filings Ser. Nos. 13/893,405, 13/893,735, and 13/893,634.

In order to enhance softness perceptions of the laminate, nonwovens may be treated by hydrojet impingement, which may also be known as hydroenhancement, hydroentanglement or hydroengorgement. Such nonwovens and processes are described in, for example, U.S. Pat. Nos. 6,632,385 and 6,803,103, and U.S. Pat. App. Pub. No. 2006/0057921, the disclosures of which are incorporated herein by reference.

Other examples of nonwoven web that may be useful in the present laminate may be an SMS web (spunbond-meltblown-spunbond web) made by Avgol Nonwovens LTD, Tel Aviv, Israel, under the designation XL-S70-26; a softband SSS (spunbond-spunbond-spunbond) web made by Pegas Nonwovens AS in Znojmo, Czech Republic, under the designation 18 XX 01 00 01 00 (where XX=the variable basis weight); an SSS web made by Gulsan Sentetik Dok San VE TIC AS, in Gaziantep, Turkey, under the designation SBXXF0YYY (where XX=the variable basis weight, and YYY=the variable cross direction width); an HESB (hydroenhanced spunbond) web made by First Quality Nonwovens Inc., in Hazelton, Pa., under the designation SEH2503XXX (where XXX=the variable cross direction width); and a bicomponent SS web.

A nonwoven web useful as a component to form one or both of layers may be pre-bonded, prior to aperturing as described below. A batt of fibers may be calendered and pre-bonded in a pattern, to consolidate the batt/fibers and create a pattern of bonds that adds tensile strength and dimensional stability, converting the batt of fibers to a coherent and useable nonwoven web material. The web may be imparted with a pattern of pre-bonding as described in, for example, U.S. Pat. No. 5,916,661 (pre-bonding in a pattern of “point calendered bonds 200 to form a coherent web structure”) and co-pending U.S. application Ser. No. 13/893,405 (pattern of “primary fiber bonds”). The pre-bonding may consist of a pattern of thermal bonds, mechanical bonds or adhesive bonds, although in some circumstances thermal bonding may be preferred.

The nonwovens described herein may be used for the belt nonwovens, including for the belt outer nonwoven and/or the belt inner nonwoven. The nonwovens may also be used for other article components, such as topsheet, backsheet, wrapping layer, outer cover, or specific subcomponents, such as landing zones, flaps, etc.

### Apertured Nonwovens

The nonwoven layers of the belt may be apertured. Using a nonwoven web that has been apertured in the manner described below to form one or both of nonwoven web layers in a belt as described above can provide attractive and interesting effects. The apertures and the material surrounding them interact with the contraction-induced rugosities in the web layer as the belt is moved and stretched as, for example, during wear. Apertures in a layer will open, close, change shape and shift relative the other layer, providing a visual impression of complexity, depth and added texture.

An example of a process for creating apertures in a pre-bonded nonwoven web is described in U.S. Pat. Nos. 5,916,661 and 5,629,097. This process involves rolling the pre-bonded nonwoven web through the nip between a pair of rollers, one of which bears a pattern of raised bonding protrusions, and supplying heating energy to heat the fibers beneath the protrusions in the nip. When appropriately controlled pressure and heating energy are provided at the nip, a pattern of suitable bonds or “weakened, melt-stabilized locations” having rod shapes or other shapes results. At the bond sites, the polymer fibers of the web are melted, compressed and thereby fused, such that the fused polymer material at the bond sites is relatively thin (in the z-direction) and frangible. Upon subsequent cross direction incremental stretching of the bonded nonwoven web as described in the above-cited patents, the material at the bond sites or “melt-stabilized locations” breaks and apertures open in a direction transverse to the long dimension of the rod shapes. For example, a nonwoven web may be thermal/calender bonded with a bonding pattern of rod shapes having their long dimension oriented in the machine direction. Following such bonding, the web may be subjected to an incremental stretching process to stretch the web in the cross direction. When the bonding process has been appropriately controlled to create relatively thin, frangible bond sites, this causes the rod-shaped bonds to break open, creating apertures through the web. Advantageously, fibers of the nonwoven web along the edges of the apertures are fused as a result of the bonding process. In comparison to a process in which apertures are simply punched or cut through the web without application of heating energy, the bonding/stretching process described in the above-cited patent does not cut the fibers, which can result in loose fibers and fraying about the edges of the punched or cut apertures. In contrast, the bonding/stretching process described herein tends not to create loose fibers, and provides more neatly defined edges about the apertures. Following incremental stretching, the web may be allowed to relax, which may cause the apertures to close to some extent, but they will still be present. Processes for aperturing may be additionally found in U.S. filings Ser. Nos. 14/032,595 and 14/032,622.

In another example, the web may be bonded by compression bonding without the application of externally-produced or additional heating energy. Examples of suitable compression bonding systems utilizing rollers are described in, for example, U.S. Pat. Nos. 4,854,984 and 4,919,738. In these types of mechanisms, a first roller and second roller are arranged with their axes in parallel and urged together to form a nip. The first roller may have on its surface one or more bonding protrusions arranged in a pattern. The first roller and second roller may be urged together by one or more actuators such as bellows-type pneumatic actuators acting directly or indirectly on one or both of their axles, to provide and regulate compression, beneath the protrusions at the nip, of the web material as it passes therethrough, in the manner described in the aforementioned patents. A compression bonding mechanism such as, but not limited to, the mechanism described in the aforementioned patents, provides bonding of a nonwoven web material through rapid compression of superimposed fibers beneath the bonding protrusions, along the roller nip line. Without intending to be bound by theory, it is believed that rapid compression beneath the protrusions causes the respective materials to be rapidly deformed and partially expressed together from beneath the protrusions, to form structures of deformed, compressed and entangled fiber material beneath and/or around the protrusions. Welds or weld-like structures at or about the protrusions result. In some circumstances compression bonding provides advantages, including relative simplicity and cost effectiveness. It may reduce or eliminate the need for more complex bonding systems that require a system to supply externally produced or additional heating energy. Without intending to be bound by theory, it is believed that these advantages are substantially independent of variations in line speeds in at least some circumstances, including line speeds within currently known economically and technically feasible ranges for manufacture of disposable diapers and training pants. Following such creation of compression bonds, the web may be incrementally stretched to create apertures at the bond sites, in the manner taught by U.S. Pat. No. 5,916,661.

As noted, as suggested in U.S. Pat. No. 5,916,661, prior to aperturing, the nonwoven web may be pre-bonded with a relatively dense pattern of thermal/calender bonds. Following that, a pattern of apertures may simply be punched or cut through the web. A relatively dense pattern of bonding can serve to minimize loose cut fibers and fraying, and help maintain defined edges of apertures formed by cutting or punching.

It will be appreciated that the apertures created need not necessarily be rod-shaped. Other examples of shapes and patterns are described in co-pending provisional application Ser. No. 61/820,987. Also, the apertures may be rod-shaped, arc-shaped, other curved finite paths, circular, oval, elliptical or polygon, and any combinations thereof. It may be desired in some circumstances, however, that the longest dimension of a majority of the individual apertures be oriented along the machine direction of the nonwoven web—particularly when the web or components of it are formed by processes that produce a machine direction bias in the fibers such as spunbonding or spunlaying processes. (For purposes herein, “oriented along the machine direction” means that the machine direction vector component of the longest dimension of an aperture is greater than the cross direction vector component.) Because of such fiber orientation, this reduces chances that sections of fibers between adjacent apertures along the machine direction will fray or tear away. At the same time, however, while it may be desired in some circumstances that the longest dimension of a majority of the apertures be oriented along the machine direction, it may also be desired that the longest dimension is not parallel with the machine direction. In one example in which the apertures are elliptical or oval-shaped, it may be desired that their longest dimensions are oriented along angle(s) a between greater than 0 and less than 45 degrees of the machine direction. It will be appreciated that this may add to visual and actual texturing effects, by causing the material along the edges of the apertures to move in a more complex manner in the machine, cross and z-directions as the belt is stretched and moved as during wear. It will also be appreciated that the apertures may be arranged in varying patterns, such as but not limited evenly-spaced and aligned rows and columns, offset rows and columns, diagonal patterns, shaped patterns, etc.

Additionally, the pattern of the apertures may be substantially similar or identical to the pattern of the pre-bonds (if present), in one or more of machine-direction spacing, cross-direction spacing, aperture shape and aperture size. For example, a pattern of pre-bonds may have substantially similar machine and cross direction spacing as the pattern of apertures. Using respective patterns of pre-bonds and apertures that are substantially similar in one or more respects noted can help give the material a more uniform, orderly and/or coherent appearance, and may also help enhance tensile strength as compared with a web in which respective patterns of pre-bonds and apertures do not have such similarities.

Using a nonwoven web that has been apertured in the manner described above to form one or both of nonwoven web layers in a belt as described above, can provide attractive and interesting effects. The apertures and the material surrounding them interact with rugosities in the web layer, providing a visual impression of complexity, depth and added texture. Apertures with various shapes, and angles relative the machine direction, can result in z-direction projections and/or ridges along the edges of the apertures when the belt structure contracts. Examples of patterns may be found in U.S. filings Ser. Nos. 14/032,595 and 14/032,622.

For example, when the belt structure contracts in either the lateral or longitudinal direction, “flaps” created by the depicted aperture shapes may stand up and add z-direction loft in addition to the height of the rugosities. The added loft may contribute to tactile and visual perceptions of added softness and/or breathability. Additionally, with expansion and contraction of the belt structure the “flaps” may open and close, alternately revealing and concealing any contrasting appearance and/or color of the underlying layer, and giving the belt structure a more complex and lively appearance.

It may be appreciated that the pattern of apertures selected may be coordinated with the pattern of adhesive selected to adhere the laminate, for varying effects. Again, see U.S. filings Ser. Nos. 14/032,595 and 14/032,622 for examples of adhesive patterns.

For example, a pattern of apertures may be selected that is somewhat independent of the pattern of rugosities created by a pattern of adhesive. The adhesive pattern may be selected so as to provide, for example, orderly machine direction rows but disordered or random cross direction columns of rugosities. The pattern of apertures may be sized and ordered so as to fall randomly on the rugosities in the machine and/or cross directions. As a result, the apertures will be positioned relative the rugosities in a somewhat random fashion, providing a particular visual effect. In another example, the pattern of adhesive may be selected to provide substantially orderly machine direction rows and cross-direction columns of rugosities. The apertures may be patterned, for example, so as to cause them to fall on the peaks of the rugosities, in, for example, substantially evenly-spaced rows and substantially evenly-distributed numbers. In this latter example, the apertures are positioned substantially at the peaks of the rugosities at a location on the nonwoven web layer at which they will experience the most movement (having another visual effect), as the belt is stretched and moved, as during wear of the article. Similarly, the apertures may be patterned in coordination with the spacing between the elastic members such as strands, such that they are substantially evenly distributed relative the locations of the strands in the belt. For example, a pattern of apertures may have an aperture spacing ASC along the cross direction that is a substantially even multiple or substantially even divisor of the elastic band spacing ESC in the cross direction in any given portion, or even the entirety, of the belt. Similarly, a pattern of apertures may have an aperture spacing ASM along the machine direction that is a substantially even multiple or substantially even divisor of the rugosity spacing RSM in the machine direction in any given portion, or even the entirety, of the belt. In one example, RSM≈ASM, so each divides into the other substantially evenly by 1. In the same example, ESC≈3 ASC, so ESC divides substantially evenly into ASC by 3. Another example may be wherein the pattern of adhesive deposits is configured to produce rugosities with peaks and valleys that are substantially continuous along the cross direction (i.e., having few or no interruptions at the elastic strands or elsewhere); this may be accomplished by a pattern of substantially continuous, linear deposits of adhesive between layers along the cross direction. The pattern of apertures may be configured such that the aperture spacing ASM along the machine direction is a substantially even multiple or substantially even divisor of the rugosity spacing RSM in the machine direction.

The visual effects of an apertured nonwoven layer in a belt may be multiplied if both layers are apertured. In the event aperturing of both layers is desired, however, it may be desirable that that the apertures of the respective layers are offset relative each other, in other words, that they do not align when the material is stretched against elastic-induced contraction to pull out the rugosities. This may be deemed important to avoid giving the belt a ragged appearance, or to avoid portions of the wearer's skin showing through the belt, or both. For this reason, it may be desired that the spacing of the aperture patterns in the respective layers differ. Alternatively, where substantially identical aperture patterns are present in both layers, it may be desired that the patterns are not in phase with each other in either or both the machine direction and cross direction, when the belt is stretched against elastic-induced contraction to pull out the rugosities.

The visual effects of an apertured nonwoven layer in a belt may be enhanced if the material of one layer has a color that visually contrasts with the color of the other layer. The material of one or both layers may be tinted, pigmented or printed in one or more colors or shades (including white) such that the colors or shades of the respective layers visually contrast. The contrasting color or shade of one layer can then be seen through apertures of the other layer for interesting visual effect. Herein, a “visual contrast” between colors or shades of two respective layers of material means that the value of delta E* determined through the Visual Contrast method, described in U.S. filings Ser. Nos. 14/032,595 and 14/032,622, is equal to or greater than 2.0. For enhanced visual contrast, it may be preferred that the value of delta E* be equal to or greater than 3.5.

Nonwoven web materials of the type typically used to form such belts are generally highly breathable. (Breathability, typically reflected in measurable vapor permeability of the material, is desired to avoid overhydration of the wearer's skin beneath the article.) Accordingly, it not necessary or desirable to provide apertures merely for the purpose of increasing breathability. Because the materials are already highly breathable aperturing may have little effect in this regard. However, it is believed that the visible presence of apertures in the material may in some circumstances give consumers the impression of high breathability, or reinforce or increase such impression—which may provide a marketing advantage for the manufacturer.

### Top Sheet

In one embodiment, the absorbent article may comprise a topsheet. The topsheet may be compliant, soft feeling, and non-irritating to the wearer's skin and may be elastically stretchable in one or more directions. Further, the topsheet may be liquid pervious, permitting liquids (e.g., menses, urine, and/or runny feces) to penetrate through its thickness. Various topsheets may also comprise a hydrophilic material, for example, which is configured to draw bodily fluids into an absorbent core of the chassis when these fluids are expelled from the body. A suitable topsheet may be manufactured from a wide range of materials, such as woven and nonwoven materials, apertured or hydroformed thermoplastic films, apertured nonwovens, porous foams, reticulated foams, reticulated thermoplastic films, and/or thermoplastic scrims, for example. Suitable apertured films may comprise those described in U.S. Pat. Nos. 3,929,135, 4,324,246, 4,342,314, 4,463,045, 5,006,394, 5,628,097, 5,916,661, 6,545,197, and 6,107,539.

Apertured film or nonwoven topsheets typically may be pervious to bodily exudates, yet non-absorbent, and have a reduced tendency to allow fluids to pass back through and rewet the wearer's skin. Suitable woven and nonwoven materials may comprise natural fibers, such as, for example, wood or cotton fibers, synthetic fibers, such as, for example, polyester, polypropylene, or polyethylene fibers, or combinations thereof. If the topsheet comprises fibers, the fibers may be spunbond, carded, wet-laid, meltblown, hydroentangled, or otherwise processed, for example, as is generally known in the art.

The topsheet may comprise a skin care lotion. Examples of suitable lotions include, but are not limited to, those described in U.S. Pat. Nos. 5,607,760; 5,609,587; 5,635,191; 5,643,588; and 5,968,025, and as described in U.S. Application No. 61/391,353, and as described in U.S. Pub. No. 2014-0257216. Beyond these compositions, the absorbent article may comprise soluble cyclodextrin derivatives such as those described in U.S. Pub. No. 2014/0274870.

Additionally, the topsheet of the present disclosure may be a tufted laminate web as disclosed in U.S. Pat. No. 7,410,683, and/or may be an apertured web as disclosed in PCT/CN2014/083769 having an international filing date of Aug. 6, 2014.

In one embodiment, the topsheet may comprise graphics such that depth perception is created as described in U.S. Pat. No. 7,163,528. In other embodiments, the topsheet may be an integrated acquisition layer and topsheet as described in U.S. Ser. No. 14/680,426 or 14/634,928.

### Backsheet

In one embodiment, the absorbent article may comprise a backsheet. The backsheet may be impervious, or at least partially impervious, to fluids or body exudates (e.g., menses, urine, and/or runny feces) and may be manufactured from a thin plastic film, although other flexible liquid impervious materials may also be used. The backsheet may prevent the body exudates or fluids absorbed and contained in an absorbent core of the absorbent article from wetting articles which contact the absorbent article, such as bedsheets, pajamas, clothes, and/or undergarments. The backsheet may comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). A suitable backsheet may comprise a polyethylene film having a thickness of from about 0.012 mm (0.5 mils) to about 0.051 mm (2.0 mils). Examples of polyethylene films are manufactured by Clopay Corporation of Cincinnati, Ohio, under the designation BR-120 and BR-121, and by Tredegar Film Products of Terre Haute, Ind., under the designation XP-39385.

One suitable material for the backsheet can be a liquid impervious thermoplastic film having a thickness of from about 0.012 mm (0.50 mil) to about 0.051 mm (2.0 mils), for example including polyethylene or polypropylene. Typically, the backsheet can have a basis weight of from about 5 g/m2 to about 35 g/m2. The backsheet can be typically positioned adjacent the outer-facing surface of the absorbent core and can be joined thereto. For example, the backsheet may be secured to the absorbent core by a uniform continuous layer of adhesive, a patterned layer of adhesive, or an array of separate lines, spirals, or spots of adhesive. Illustrative, but non-limiting adhesives, include adhesives manufactured by H. B. Fuller Company of St. Paul, Minn., U.S.A., and marketed as HL-1358J. An example of a suitable attachment device including an open pattern network of filaments of adhesive is disclosed in U.S. Pat. No. 4,573,986. Another suitable attachment device including several lines of adhesive filaments swirled into a spiral pattern is illustrated by the apparatus and methods shown in U.S. Pat. Nos. 3,911,173; 4,785,996; and 4,842,666. Alternatively, the attachment device may include heat bonds, pressure bonds, ultrasonic bonds, dynamic mechanical bonds, or any other suitable attachment device or combinations of these attachment devices.

In one embodiment, the backsheet may be embossed and/or matte-finished to provide a more cloth-like appearance. Further, the backsheet may permit vapors to escape from the absorbent core of the absorbent article (i.e., the backsheet is breathable) while still preventing, or at least inhibiting, fluids or body exudates from passing through the backsheet. In one embodiment, the size of the backsheet may be dictated by the size of the absorbent article and the design or configuration of the absorbent article to be formed, for example.

### Absorbent Core

In various embodiments, the absorbent article may comprise an absorbent core (also referred to as an “absorbent member” or “absorbent assembly” or “absorbent structure” or “absorbent composite”) that is disposed between the topsheet and the backsheet. The absorbent core may comprise a laterally extending front edge in the front waist region, a longitudinally opposing and laterally extending back edge in the back waist region, a first longitudinally extending side edge, and a laterally opposing and second longitudinally extending side edge. Both of the side edges may extend longitudinally between the front edge and the back edge. In one embodiment, more than one absorbent core or more than one absorbent core layer may be provided in an absorbent article, for example. The absorbent core may be any suitable size or shape that is compatible with the absorbent article. Example absorbent structures for use as the absorbent core of the present disclosure that have achieved acceptance and commercial success are described in U.S. Pat. Nos. 4,610,678; 4,673,402; 4,888,231; and 4,834,735.

In one embodiment, suitable absorbent cores may comprise cellulosic airfelt material (also referred to as pulp). For instance, such absorbent cores may comprise less than about 40%, 30%, 20%, 10%, 5%, or even 1% of the cellulosic airfelt material as determined by weight. Additionally, such an absorbent core may be primarily comprised of an absorbent gelling material (AGM) in amounts of at least about 60%, 70%, 80%, 85%, 90%, 95%, or even about 100% as determined by weight. Furthermore, a portion of the absorbent core may comprise a microfiber glue (if applicable). Such absorbent cores, microfiber glues, and absorbent gelling materials are described in U.S. Pat. Nos. 5,599,335; 5,562,646; 5,669,894; 6,790,798; and 7,521,587 and in U.S. Pat. Publ. No. 2004/0158212.

In one embodiment, the core, including multiple layers making up the core system, may be printed and embossed as described in U.S. Pat. No. 8,536,401.

In one embodiment, the core may be separable from the chassis as disclosed in U.S. Pat. Nos. 6,989,006; 7,381,202; 7,175,613; 7,824,386; 7,766,887; and 6,989,005. In such embodiments, the measurements described in this disclosure may be made to the chassis alone or may be made to the chassis in combination with the separable core/absorbent assembly.

In one embodiment, the absorbent article of the present disclosure, and particularly, a portion where the absorbent member is disposed, may have a body fluid absorption rate greater than 3 g/sec according to U.S. Pat. No. 6,649,810. According to U.S. Pat. No. 6,649,810, the expression “the portion (of the absorbent article) where the absorbent member is disposed” is intended to mean the portion occupied by the absorbent member when the absorbent article is flatly unfolded and seen in its plan view.

In one embodiment, the absorbent structure may have an intake factor greater than 3 according to U.S. Pat. No. 7,073,373, wherein the intake factor is defined as the absorbent core permeability divided by the normalized retention capacity (which is defined by the Retention Capacity Test—also according to U.S. Pat. No. 7,073,373). In one embodiment, the absorbent composite has a body fluid absorption greater than 75 g/100 cm2, according to U.S. Pat. No. 6,649,810.

In one embodiment, a target location of the absorbent article may have a wicking value greater than 36%, according to U.S. Pat. No. 6,383,960.

In one embodiment, the absorbent article may have a bending stiffness between 0.05-1.0 gf, according to U.S. Pat. No. 5,810,796.

In one embodiment, the absorbent article may have a crotch fluid absorption rate greater than 3 g/sec according to U.S. Pat. No. 6,649,810. In one embodiment, a freeze-dried composite of the absorbent composite may have an intake rate of at least about 1.9 cubic centimeters (cc) of liquid/second at 80% composite saturation according to U.S. Pat. No. 6,689,934.

In some embodiments the absorbent core may comprise channels as described in U.S. Pat. No. 8,568,566; U.S. Pub. Nos. 2012/316046, 2014/027066, 2014/163500, 2014/163506, 2014/163511, 2012/316526, 2012/316527, 2012/316528, 2012/316529, 2012/316523, 2014/163501, 2014/163502, 2014/163503 and European Pub. Nos. 2532328, 2532329, 2717823, 2717820, 2717821, 2717822, 2532332, 2740449, and 2740452.

In some embodiments the absorbent layer may comprise at least two channels substantially free of absorbent polymer particles extending through the thickness of the absorbent layer in the longitudinal dimension of the absorbent layer. By extending in the longitudinal dimension of the absorbent layer, it is meant that the channels extend essentially in the longitudinal dimension, i.e. they extend more in the longitudinal dimension than in the transverse dimension, e.g. at least twice as much in the longitudinal dimension than in the transverse dimension.

“Channels” as used herein refer to discrete portions of one or more of the absorbent layers of the absorbent core extending through the thickness of the absorbent layer which are substantially free of absorbent polymer (particles or fibers), i.e., no absorbent polymer particles are intentionally present in such a channel (longitudinal main channel or secondary channel) of an absorbent structure. However, it should be understood that, accidentally, a small, negligible amount of absorbent polymer particles may be present in the channel, which may not contribute to any significant degree to the overall functionality (e.g. absorbency of the absorbent structure). Typically, the channels possess two transverse edges (in the shortest dimension) and two longitudinal edges (in the longest dimension) running between the transverse edges. The transverse edges of the channels may be straight (i.e., perpendicular to the longitudinal side edges), angled or curved. The channels may have an average width w of at least 3 mm (the average of a channel is defined as the average distance between the longitudinal side edges) or may have at least 4% of the width of the absorbent layer.

The channels may be permanent. By permanent, it is meant that the integrity of the channels is at least partially maintained both in dry state and wet state, i.e., the channels are resistant to external forces caused by movements of the diaper's wearer. Permanent channels are obtained by immobilizing the absorbent polymer on the substrate layer, such as by applying a thermoplastic adhesive material over the absorbent layer. The absorbent layer of the present disclosure may comprise in particular permanent channels formed by bonding of a first substrate layer and a second substrate layer through the channels. Typically, glue may be used to bond both substrate layers through the channel, but it is possible to bond via other known means, for example ultrasonic bonding, pressure bonding or thermal bonding. The supporting layers can be continuously bonded or intermittently bonded within the channels.

In some embodiments, it may be desirable to have an array of articles comprising absorbent cores with channels, such as those disclosed in 62/104,330.

### Leg Cuffs

In one embodiment, the chassis of the absorbent article may comprise longitudinally extending and laterally opposing leg cuffs and that are disposed on the interior surface of the chassis that faces inwardly toward the wearer and contacts the wearer. The leg cuffs and may comprise one or more elastic gathering members disposed at or adjacent the proximal edge of one or both of the leg cuffs. In addition, the elastic gathering members of the leg cuff may also comprise one or more elastic strands disposed at or adjacent the distal edge of one or both of the leg cuffs. The elasticized leg cuffs may comprise several embodiments for reducing the leakage of body exudates or fluids in the leg regions. The elasticized leg cuffs are sometimes referred to as leg bands, barrier cuffs, elastic cuffs, or gasketing cuffs. Suitable elasticized leg cuffs may comprise those described in U.S. Pat. Nos. 3,860,003, 4,909,803, 4,695,278, 4,795,454, 4,704,115, and 4,909,803, and U.S. Pat. Publ. No. 2009/0312730. The leg cuffs may be formed by folding portions of the chassis laterally inward, i.e., toward the longitudinal axis, to form both the respective leg cuffs and the side edges of the chassis. In other embodiments, the leg cuffs may be formed by attaching an additional layer or layers to the chassis at or adjacent to each of the respective side edges of the chassis. In one embodiment, the chassis may also comprise other elastics disposed adjacent the side edges which may cause the article to form into a “U” shape when allowed to relax thereby pulling the interior surface of the front waist region toward the interior surface of the back waist region.

In one embodiment, each leg cuff may comprise a proximal edge. These edges are positioned proximate to the longitudinal axis compared to distal edges. The leg cuffs may overlap the absorbent core, i.e., the proximal edges lie laterally inward of the respective side edges and of the absorbent core. Such an overlapped configuration may be desirable in order to impart a more finished appearance to the absorbent article than that imparted by a non-overlapped configuration. In other embodiments, the leg cuffs may not overlap the absorbent core.

In one embodiment, each leg cuff may be attached to the interior surface of the chassis in a leg cuff attachment zone (not shown) adjacent to the front waist end edge and in a longitudinally opposing leg cuff attachment zone (not shown) adjacent to the back waist end edge. In one embodiment, between the leg cuff attachment zones, the proximal edge of the leg cuff remains free, i.e., not attached to the interior surface of the chassis or to the absorbent core. Also, between the longitudinally opposing leg cuff attachment zones, each leg cuff may comprise one or more (specifically including one, two, three, or four elastic strands per leg cuff) longitudinally extensible cuff elastic gathering members that may be disposed at or adjacent to the proximal edge of the leg cuff by any suitable methods. Each of such cuff elastic gathering members may be attached over the leg cuff's entire length or over only a portion of the leg cuff's length. For example, such cuff elastic gathering members may be attached only at or near the leg cuff's longitudinally opposing ends and may be unattached at the middle of the leg cuff's length. Such cuff elastic gathering members may be disposed in the crotch region and may extend into one or both of the front waist region and the back waist region. For example, an elastic gathering member may be attached at or adjacent to the proximal edge of each of the leg cuffs and extends into both the front waist region and the back waist region.

In various embodiments, each cuff elastic gathering member may be enclosed inside a folded hem for example. In various embodiments, the cuff elastic gathering members may be sandwiched between two layers forming the leg cuff, by two layers of the chassis, or may be attached on a surface of the chassis or the leg cuff and remain exposed.

In one embodiment, when stretched, the cuff elastic gathering member disposed adjacent to each leg cuff's proximal edge allows the leg cuff proximal edge to extend to the flat uncontracted length of the chassis, e.g., the length of the chassis. When allowed to relax, the cuff elastic gathering member contracts to pull the front waist region and the back waist region toward each other and, thereby, bend the article into a “U” shape in which the interior of the “U” shape may be formed by the portions of the article that are intended to be placed toward the body of the wearer (i.e., interior surface). Because each of the proximal edges remains free between the longitudinally oriented leg cuff attachment zones, the contractive force of the elastic gathering member may lift the proximal edge of the leg cuff away from the interior surface of the chassis. This lifting of the proximal edges when the article is in the relaxed condition lifts the leg cuffs into a position to serve as side barriers to prevent, or at least inhibit, leakage of bodily exudates.

Examples of acceptable leg cuffs are disclosed in U.S. Ser. No. 13/457,521, filed Apr. 27, 2012, including the configurations disclosed by FIGS. 8a-t of the '521 application.

### Waistband

In one embodiment, the article may comprise an elasticized waistband. The elasticized waistband may provide improved fit and containment and may be configured to elastically expand and contract laterally to dynamically fit a wearer's waist. The elasticized waistband may extend longitudinally from the waist edge of the absorbent article toward the waist edge of the absorbent core. In one embodiment, the absorbent article may have two elasticized waistbbands, one positioned in the back waist region and one positioned in the front waist region, although other pant embodiments may be constructed with a single elasticized waistband. The elasticized waistband may be constructed in a number of different configurations including those described in U.S. Pat. Nos. 4,515,595 and 5,151,092, and including the consolidated gathers as disclosed in U.S. Ser. Nos. 13/490,543, 13/490,548, and 13/490,554.

In one embodiment, the elasticized waistbands may comprise materials that have been “prestrained” or “mechanically prestrained” (i.e., subjected to some degree of localized pattern mechanical stretching to permanently elongate the material). The materials may be prestrained using suitable deep embossing techniques. In other embodiments, the materials may be prestrained by directing the material through an incremental mechanical stretching system as described in U.S. Pat. No. 5,330,458. The materials may then be allowed to return to their substantially untensioned condition, thus forming a zero strain stretch material that is extensible, at least up to the point of initial stretching. Examples of zero strain materials are disclosed in U.S. Pat. Nos. 2,075,189, 3,025,199, 4,107,364, 4,209,563, 4,834,741, and 5,151,092.

### Flaps

The flaps may be discrete from or integral with the chassis. A discrete flap is formed as separate element, which is joined to the chassis. In some embodiments this may include a front and/or back belt-like flaps (“belts”) being joined across the front and back (or rear) waist regions of the chassis, at least across end edges of the chassis. In some embodiments the waistbands can overlap the flaps to create a continuous belt-like structure.

The belt-like flaps may comprise an inner nonwoven layer and an outer nonwoven layer and elastics there between. The inner and outer nonwoven layers may be joined using adhesive or thermoplastic bonds. Various suitable belt-like flap configurations can be found in U.S. Pub. No. 2013-0211363.

An integral flap is a portion, one or more layers, of the chassis that projects laterally outward from the longitudinal edge. The integral flap may be formed by cutting the chassis to include the shape of the flap projection.

While many of the embodiments illustrated in this application having belt-like flaps are pant articles, taped articles may have belt-like flaps disposed in one or both waist regions as well.

The structure of flaps play an important role in the functionality of the absorbent article and are fundamentally different than the elastics used in underwear. In order to sustain the fit of the article even after loading the article comprises elastomeric element(s), including films and/or strands that are disposed proximate to and along the side seams of the article and extend laterally from one side toward the other. These elastomeric element(s) should create a normal force against the body sufficient to anchor the article. The location of the elastomeric element(s), as well as the forces exerted by the elastomeric element(s) can be varied to ensure proper anchoring at the hips and along the body specifically across the front waist region and in the back waist region. One form of anchoring beneficial for sustaining the fit of a loaded article is disclosed in U.S. Pat. No. 5,358,500 Absorbent Articles Providing Sustained Dynamic Fit issued Oct. 25, 1994 to LaVon, et al.

The seams may each be from about 70 mm to about 200 mm, from about 100 mm to about 190 mm, or from about 130 mm to about 150 mm. The seams are the portions of the flap that overlap (i.e., the distance from the waist opening to the leg opening of the overlapped or abutted flaps).

### Fastening System

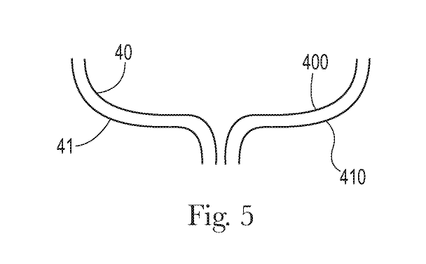

The absorbent article may also include a fastening system. When fastened, the fastening system interconnects the front waist region and the rear waist region resulting in a waist circumference that may encircle the wearer during wear of the absorbent article. The fastened elements connecting the front and back waist regions form refastenable side seams. This may be accomplished by flaps in the back waist region interconnecting with flaps in the front waist region or by flaps in the back waist region interconnecting with the chassis in the front waist region. The fastening system may comprises a fastener such as tape tabs, hook and loop fastening components, interlocking fasteners such as tabs & slots, buckles, buttons, snaps, and/or hermaphroditic fastening components, although any other known fastening means are generally acceptable. The fasteners may releasably engage with a landing zone, which may be a woven or nonwoven. Some exemplary surface fastening systems are disclosed in U.S. Pat. Nos. 3,848,594; 4,662,875; 4,846,815; 4,894,060; 4,946,527; 5,151,092; 5,221,274. Particularly, the flaps may be configured as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of the '065 application). An exemplary interlocking fastening system is disclosed in U.S. Pat. No. 6,432,098. The fastening system may also provide a means for holding the article in a disposal configuration as disclosed in U.S. Pat. No. 4,963,140. The fastening system may also include primary and secondary fastening systems, as disclosed in U.S. Pat. No. 4,699,622. The fastening system may be constructed to reduce shifting of overlapped portions or to improve fit as disclosed in U.S. Pat. Nos. 5,242,436; 5,499,978; 5,507,736; and 5,591,152.

In some embodiments, a refastenable system may be used such as those disclosed in U.S. applications Ser. Nos. 13/929,900, 13/929,970. The particular hooks (types and sizes) and landing zones disclosed in 62/063,445 may also be used.

The embodiment shown in FIG. 2 comprises fastening elements 201-204 that may be refastenably joined together. Particularly, fastening elements 201 and 203 may be hook elements that join with fastening elements 202 and 204, respectively. Fastening elements 201 and 203 are shown on an exterior surface of the elasticized belt 30, but they may also be placed on an interior surface of the elasticized belt 30. Fastening elements 202 and 204 may be a discrete member of loop elements or may be an area of loop elements that is part of a nonwoven sheet lining the interior (as shown) or exterior of the elasticized belt. In another embodiment, fastening elements 201 and 203 may be loop elements and fastening elements 202 and 204 may be hook elements.

It is understood that when the fastening elements 201-204 mate interior surface to interior surface of the elasticized belt 30, a flange seam is formed. This may be a permanent side seam. One embodiment is shown in FIG. 5. FIG. 5 shows the rear portion inner belt nonwoven 40 coming together with the front portion inner belt nonwoven 400, looking at the article from the top. The interiors of both inner belts are bonded, forming a permanent seam. Both the front and rear portion inner belts 40 and 400 may be made of PP (polypropylene), so the PP to PP seam provides a bond with good strength. Both front and rear outer belts 41 and 410 may be made of PE/PET (polyethylene/polyethylene terephthalate) core/sheath bicomponent material, wherein the core is the PET and the outer sheath is PE.

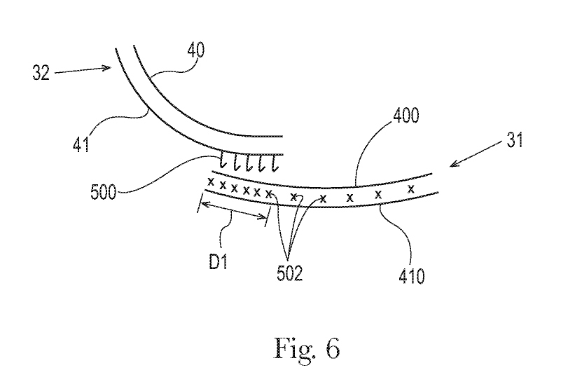

When the fastening elements 201-204 mate interior surface to exterior surface of the elasticized belt 30, an overlap seam is formed. An example of this is shown in FIG. 6. FIG. 6 shows a top view of the area where the rear belt 32 is overlapped by the front belt 31, forming a refastenable seam. Specifically, the rear portion outer belt nonwoven 41 is overlapped by, ie., brought together with, the front portion inner belt nonwoven 400. The rear portion outer belt nonwoven may be a blend of PE/PET (as described above), while the front portion inner belt nonwoven may be made of PP. In FIG. 6, the rear portion outer belt nonwoven has an area comprising hooks 500. In general, the hooks may be disposed on the outer surface of a belt and face away from the wearer and connect into the inner layer of the opposing belt, or connect into a landing zone disposed on the inner layer of the opposing belt. In some cases, the hooks may be attached to the nonwoven with more than one type of bonding, for example, an adhesive plus pressure bonding. In some cases, the bonding may be effective to bond the hooks 500, the outer belt nonwoven 41 and the inner belt nonwoven 40 to assure that the force exerted on the hook 500 does not overcome the bond between the hook and the outer belt nonwoven, and the bond between the outer belt nonwoven and the inner belt nonwoven. In FIG. 6, the polypropylene of the front portion inner belt nonwoven may provide a good landing zone, where the hooks directly connect with the fibers of the front portion inner belt nonwoven. Or, there can be an additional landing zone with loops, where the landing zone is attached to the front portion inner belt nonwoven.

The front or rear belt portion that is used as the landing zone may also have an area of additional bonding between its outer and inner belts to assure adequate strength as a landing zone. That is, a front or rear belt portion that is acting as a landing zone must have a strong enough bond between its inner and outer belts to hold when the front or rear belt portion is attached to the opposite belt portion. Therefore, as shown in FIG. 6, there is bonding 502 between the inner and outer belts of the front belt portion. There may be additional bonding (e.g., more adhesive, higher ultrasonic frequency bonding, and/or more pressure bonding, etc.) for a length from about 10 mm to about 45 mm, D1, to assure that the force exerted on the front belt portion as a landing zone does not overcome the bond between the front belt portion's inner and outer belts. The bonding between the front belt portion inner and outer belts may be less outside of the length of D1, as, in general, less bonding allows the material to be softer.

The fastening elements 201-204, first and second fastening elements 202 and 204 and first and second mating fastening elements 201 and 203, may be fastened during the manufacturing process and/or fastened in the package prior to use by the wearer or caregiver (i.e., the pant may be sold in “closed form”). Alternatively, the pant may be sold in “open form,” where the fastening elements 201-204 are present but are not joined in the package.

### Graphic Objects

Absorbent article components may comprise one or more graphics, and may more particularly be referred to as “graphic absorbent article components”. Graphics may include, but are not limited to, letters, numbers, symbols, icons, mammal representations, animal representations, insect representations, fish representations, vehicle representations, geometric shapes (e.g., circles, triangles, squares, rectangles, straight and wavy lines, etc.), animations, photographic images, plant representations, landscape representations, patterns (symmetrical or random), textile-like prints or patterns, foliage representations, anthropomorphic representations, as well as those graphics described in U.S. Pat. Pub. No. 2006/0247594. Additionally, graphics may be instructional.

Graphics may be applied to absorbent article components using a number of printing techniques and processes, including, but not limited to, relief printing (including letterpress and flexography), planographic printing (including offset lithography, screenless lithography, collotype, and waterless printing), intaglio printing (including gravure, steel-die, and copper-plate engraving), stencil and screen printing, and electronic printing (including electrostatic, magnetographic, ion or electron deposition, and ink-jet printing). Graphics may be applied to absorbent article components in the absorbent article component's relaxed or stretched state (in the case of stretchable, elastic, or extensible graphic absorbent article components, as further described in U.S. Pat. No. 5,612,118.

In some embodiments, a graphic object may be a wetness indicator, such as those described in U.S. filings Ser. Nos. 14/037,404, 62/147,258, 14/819,501, and 14/663,480. Wetness indicators may be printed on the inner surface (absorbent core side) of the impermeable layer (backsheet film).

It may be desirable for embodiments to have graphic objects such as those disclosed in U.S. application Ser. No. 11/999,229 or 62/204,680. Specifically, the patterns and arrangements of the graphic objects disclosed in U.S. Ser. No. 11/999,229 or the placement of graphics disclosed in U.S. 62/204,680 may be incorporated into the present invention. The noticeability rating as described in U.S. Ser. No. 11/999,229 may also be used to measure the seam noticeability with the particular graphic objects.

A graphic object may be placed on either the outer surface of the impermeable layer (film) or a backsheet nonwoven that is laminated on the impermeable layer. Alternatively, the graphic object may be printed after the backsheet nonwoven and the film are laminated.

Suitable methods of printing graphics and assembling article components with graphics may be found in U.S. applications Ser. Nos. 12/476,348, 14/635,189, 62/093,452, 62/093,516, 62/093,604, 62/093,620, 62/093,438, 62/147,004, and 62/147,006.

### Identical or Substantially Identical Chassis

As disclosed in U.S. Pub. No. 2013-0211355, it may be desirable to offer an array of packages for fitting different sized wearers, but comprising identical or substantially identical chassis. For instance, an array may comprise a first package comprising a first size of absorbent articles and a second package may comprise a second size of absorbent articles, where the first and second packages comprise identical or substantially identical chassis as described in U.S. Pub. No. 2013-0211355. More particularly, the first package may comprise a first chassis and the second package may comprise a second chassis, where each of the first and second chassis comprise the same dimensions of one or more of: core width at the lateral centerline, core width at one of the front or rear core end, a distance from a left outer cuff distal edge to a right outer cuff distal edge, a distance from a left inner cuff distal edge to a left outer cuff distal edge, a distance from a left inner cuff proximal edge to a right inner cuff proximal edge, a distance from a left inner cuff proximal edge to a left outer cuff distal edge, a free height of the inner cuff, inner cuff hem fold width, inner cuff elastics length, outer cuff elastics length, core length, and backsheet width.

Further, each of the first and second chassis may comprise identical chemical compositions of one or more of a topsheet, backsheet film, backsheet nonwoven, core super absorbent polymers, core pulp, core nonwoven, core tissue, leg cuff film, leg cuff nonwoven, super absorbent polymer adhesive, core nonwoven adhesive, leg cuff elastic adhesive, and backsheet nonwoven/film adhesive.

And, each of the first and second chassis may comprise the same basis weight of one or more of the topsheet, backsheet film, backsheet nonwoven, core super absorbent polymers, core pulp, leg cuff nonwoven, leg cuff film, super absorbent polymer adhesive, leg cuff adhesive, and backsheet nonwoven/film adhesive.

And, each of the first and second chassis may comprise compositionally identical core super absorbent polymers. The first and second chassis may have identical component cross sectional order and disposition in at least one of the front waist region, back waist region, and crotch region. The inner leg cuffs of the first and second chassis may be composed of the compositionally identical materials.

And, the core adhesives of the first and second chassis may be the same adhesive(s). The first and second chassis may comprise core super absorbent polymers that are in the same chemical class and subclass.

And, each of the first and second chassis may comprise first and second wetness indicators, respectively, and wherein the first and second wetness indicators are compositionally identical.

Further, the inner leg cuffs of the first and second chassis may have identical component cross sectional order and disposition in at least one of the front waist region, back waist region, and crotch region. The distance from the left outer cuff distal edge to a right outer cuff distal edge may the same. The distance from the left inner cuff proximal edge to left outer cuff distal edge may be the same. The distance from the left inner cuff proximal edge to the right inner cuff proximal edge is the same. The lengths of the inner and outer cuffs are the same.

In some embodiments, different size offerings in an array may have identical or substantially identical chassis as the flaps or belts may be used to enable the absorbent article to fit different sized wearers. For example, first and second absorbent articles may have identical chassis (compositionally, dimensionally, cross-sectionally), but the first article may have a different length due to disposition of the belts, such that the first article may be targeted to fit a smaller wearer than the second article. As a second example, first and second absorbent articles may have identical chassis (compositionally, dimensionally, cross-sectionally), but the first article may have a different length and/or width due to the size of the belts, such that the first article may be targeted to fit a smaller wearer than the second article.

In some embodiments, first and second absorbent articles may have identical chassis compositionally, but not dimensionally, and not cross-sectionally. In some embodiments, first and second absorbent articles may have identical chassis dimensionally, but not compositionally, and not cross-sectionally. In some embodiments, first and second absorbent articles may have identical chassis cross-sectionally, but not dimensionally, and not compositionally. In still other embodiments, first and second absorbent articles may have two, but not three of (1) compositionally, (2) dimensionally, and (3) cross-sectionally identical chassis.

### EXAMPLES

The table below shows Examples 1-6 that are belted structures with various combinations of components as described herein:

| | Ex. 1 | Ex. 2 | Ex. 3 | Ex. 4 | Ex. 5 | Ex. 6 |
|---|---|---|---|---|---|---|
| OBNW | PP | PE/PP | PE/PP | PE/PET | PE/PET | PP |
| | | Bico | Bico | bico | bico | |
| IBNW | PP | PP | PP | PP | PP | PP |
| waistfold | by | by | by | by | by | by |
| | OBNW | OBNW | OBNW | IBNW | IBNW | OBNW |
| Apertures | No | No | No | Yes | Yes | No |
| on NW | | | | (OBNW) | (OBNW) | |
| (Y/N) | | | | | | |
| elastic | round | round | round | non-round | non-round | round |
| shape | | | | | | |
| Elastic | all | all | Inter- | Majority | Majority | all |
| Adhesive | around | around | mittent | on IBNW | on IBNW | around |
| | | | | side | side | |
| offset belt | Yes | Yes | Yes | No | No | No |
| wetness | Yes | Yes | Yes | No | Yes | Yes |
| indicatior | | | | | | |
| Flange | Flange | Flange | Flange | Flange | Overlap | Flange |
| seam or | seam | seam | seam | seam | | seam |
| overlap | | | | | | |
| refas- | No | No | No | No | Yes | No |
| tenable | | | | | | |
| (Y/N) | | | | | | |
| Belt | No | Yes | Yes | Yes | Yes | Yes |
| printing/ | | | | | | |
| graphics | | | | | | |
| OBNW | 11gsm | 17gsm | 17gsm | | | |
| BW | | | | | | |
| IBNW | 10gsm | 10gsm | 10gsm | | | |
| BW | | | | | | |
| Absorbent | AGM | AGM | AGM | AGM | AGM | AGM |
| core | and | Only | Only | and | and | and |
| | Pulp | | | Pulp | pulp | Pulp |

OBNW = outer belt nonwoven
IBNW = inner belt nonwoven
BW = basis weight
Gsm = grams per square meter
Bico = bicomponent material

### Test Methods

### COF Method:

The Static and kinetic (dynamic) COF in the machine direction of the web can be measured using ASTM Method D 1894-01 with the following particulars. The test is performed on a constant rate of extension tensile tester with computer interface (a suitable instrument is the MTS Alliance using Testworks 4 Software, as available from MTS Systems Corp., Eden Prarie, Minn.) fitted with a coefficient of friction fixture and sled as described in D 1894-01 (a suitable fixture is the Coefficient of Friction Fixture and Sled available from Instron Corp., Canton, Mass.). The apparatus is configured as depicted in Figure 1c of ASTM 1894-01 using a stainless steel plane with a grind surface of 320 granulation as the target surface. A load cell is selected such that the measured forces are within 10% to 90% of the range of the cell. The tensile tester is programmed for a crosshead speed of 127 mm/min, and a total travel of 130 mm. Data is collected at a rate of 100 Hz. To obtain the specimen from a diaper, first identify the machine direction of belt nonwoven, which is typically along the lateral axis of the diaper. Carefully remove the nonwoven web layer from the diaper of sufficient size to yield a specimen. A cryogenic spray, such as CYTO-FREEZE (Control Company, Houston, Tex.), may be used to deactivate adhesives and enable easy separation of the nonwoven web layer from the underlying layer. Precondition the specimens at about 23 .deg. C.+2 C. deg. and about 50%+2% relative humidity for 2 hours prior to testing, which is performed under these same conditions.

The specimen is cut to a size of 64 mm in CD by 152 mm in MD of nonwoven specimen. Cut a 25 mm slit in the center of one of the short ends of the specimen. Place the sled on the specimen so that the 25 mm slit is aligned with the hook where the wire is connected. Pull up the slit end of the specimen so that the hook passes through the 25 mm slit, and secure the ends of the strip with tape or velcro to the top of the sled. Wrap the opposite end of the specimen around the sled without slack, but without stretching, and secure that end with tape or velcro to the top of the sled. The entire bottom surface of the sled should be covered with a continuous, smooth covering of specimen. The specimen is oriented on the sled such that the wearer-facing surface, or outward-facing surface (as on the diaperbelt) will face the target surface, and the longitudinal orientation of the specimen, is parallel to the pull direction of the sled. The mass of the sled with mounted specimen is recorded to 0.1 gram. The target surface of the stainless steel plane is cleaned with isopropanol before each test. In order to acquire CoF between nonwovens, obtain a second specimen, duplicate to the one mounted to the sled, which is large enough to cover the target surface. Place the second specimen on the target surface, oriented so that the same surface of the two specimens will face each other during the test with the machine direction parallel to the pull direction of the sled. Align the specimen on the target surface so that it is equidistant between the edges. Align the end of the specimen with the protruding end of the platform, and fix it using tape or clamps along the entire protruding end only, leaving the other end of the specimen unsecured to prevent buckling of the material during testing. The Static and Kinetic coefficients of friction (COF) for the specimen are calculated as follows:

Static COF=A[s]/B

A[s]=maximum peak force in grams force (gf) for the initial peak

B=mass of sled in grams

Kinetic COF=A[K]/B

A[K]=average peak force in grams force (gf) between 20 mm and 128 mm

B=mass of sled in grams

Testing is repeated for a total of 10 replicates of each specimen. Average and report the Static and Kinetic COF values for the replicates.

Stiffness Test

Bending Stiffness is measured in accordance with ASTM D5732-95 for nonwoven. Stiffness can be measured using an automatic F.R.L. cantilever bend tester from Testing Machines Inc. located in Ronkonkoma, N.Y. Bending stiffness is measured in MD and CD of the nonwoven. Test measures overhang length of the sample when the specimen bends and makes 41.5° angle with horizontal line under its own weight, as specified in ASTM D5732-95. Bending length is calculated from overhang length as specified in the method, and reported to the nearest 1 mm. Testing is repeated for a total of 10 replicates for CD and MD. Average bending lengths for CD and MD are reported.

All patents and patent applications (including any patents which issue thereon) referred to herein are hereby incorporated by reference to the extent that it is consistent herewith. The citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is, therefore, intended that the scope of the invention is limited only by the appended claims and equivalents thereof.

What is claimed is:

1. An absorbent article having a front region, a rear region, and a crotch region disposed therebetween; and comprising:
a liquid permeable topsheet; a backsheet, and an absorbent core disposed between the topsheet and the backsheet;
a central chassis occupying the crotch region;
a belt structure disposed about the central chassis, the belt structure overlaying the backsheet to the outside thereof in the front and rear regions, the belt structure overlapping and extending laterally and longitudinally outward from the chassis;
wherein the belt structure comprises an outer nonwoven and an inner nonwoven and elastic strands therebetween; wherein the belt outer nonwoven is a bicomponent material;
the belt structure further having a front belt portion having a front waist edge, and front left and right side edges; and a rear belt portion having a rear waist edge and rear left and right side edges;
wherein the respective front and rear left side edges and the respective front and rear right side edges are joined, forming a waist opening and left and right leg openings.

2. The absorbent article of claim 1, wherein the belt outer nonwoven has a sheath/core structure.

3. The absorbent article of claim 2, wherein sheath comprises polyethylene and the core comprises polypropylene.

4. The absorbent article of claim 2, wherein the sheath comprises polyethylene and the core comprises polyethylene terephthalate.

5. The absorbent article of claim 1, wherein the article has permanent side seams.

6. The absorbent article of claim 1, wherein the article has refastenable side seams.

7. The absorbent article of claim 6, wherein the front belt portion comprises fastening elements and the rear belt portion comprises fastening elements.

8. The absorbent article of claim 7, wherein the refastenable side seams are formed with hooks and loops.

9. The absorbent article of claim 8, wherein the front belt portion fastening elements are loops and the rear belt portion fastening elements are hooks.

10. The absorbent article of claim 6, wherein the rear outer belt portion comprises fastening elements that are hooks that fasten directly into the front belt portion inner nonwoven.

11. The absorbent article of claim 1, wherein at least one of the belt structure and the central chassis comprise a graphic object.

12. The absorbent article of claim 11, wherein at least one graphic object is printed at a place where the belt structure and the central chassis overlap.

13. The absorbent article of claim 11, wherein the central chassis comprises a graphic object that is a wetness indicator.

14. The absorbent article of claim 1, wherein the absorbent article is in a first package and wherein a second absorbent article is in a second package, and wherein the first and second articles are in closed form, wherein the first and second articles comprise different waist opening circumferences and/or product pitches, and wherein the central chassis of the absorbent article and a second central chassis of the second absorbent article are at least substantially identical.

15. The absorbent article of claim 1, wherein the absorbent article is in a first package and wherein a second absorbent article is in a second package, and wherein the first and second articles are in closed form, and wherein the absorbent article has permanent side seams and the second absorbent article comprises refastenable side seams.

16. An array of absorbent articles, comprising the first and second packages of claim 14.

17. An absorbent article having a front region, a rear region, and a crotch region disposed therebetween; and comprising:
a liquid permeable topsheet; a backsheet, and an absorbent core disposed between the topsheet and the backsheet;
a central chassis occupying the crotch region;
a belt structure disposed about the central chassis, the belt structure overlaying the backsheet to the outside thereof in the front and rear regions, the belt structure overlapping and extending laterally and longitudinally outward from the chassis;
wherein the belt structure comprises an outer nonwoven and an inner nonwoven and elastic strands therebetween; wherein the belt outer nonwoven is polyethylene;
the belt structure further having a front belt portion having a front waist edge, and front left and right side edges; and a rear belt portion having a rear waist edge and rear left and right side edges;
wherein the respective front and rear left side edges and the respective front and rear right side edges are joined, forming a waist opening and left and right leg openings.
